(19) United States
(12) Patent Application Publication
(10) Pub. No.: US 2023/0099881 A1
(43) Pub. Date: Mar. 30, 2023

(54) INTERATRIAL SHUNTING DEVICES, SYSTEMS AND METHODS

(71) Applicant: Medtronic, Inc., Minneapolis, MN (US)

(72) Inventors: Zhongping Yang, Woodbury, MN (US); Thomas A. Anderson, New Hope, MN (US); John Horrigan, Beverly, MA (US); Robert C. Kowal, Excelsior, MN (US); Brian D. Pederson, East Bethel, MN (US)

(73) Assignee: Medtronic, Inc., Minneapolis, MN (US)

(21) Appl. No.: 17/866,776

(22) Filed: Jul. 18, 2022

Related U.S. Application Data
(60) Provisional application No. 63/248,866, filed on Sep. 27, 2021.

(51) Int. Cl. A61B 17/12; A61B 17/00

(52) U.S. Cl. CPC A61B 17/12122; A61B 2017/00867; A61B 2017/00575

(57) ABSTRACT

An interatrial shunting device including a tube and an anchoring assembly. The tube defines a first end opposite a second end, and a tube wall extending to and between the first and second ends. The tube wall is a solid body and defines a lumen of the tube, with the lumen being open at the first and second ends. The anchoring assembly is carried by the tube and is configured to secure the interatrial shunting device to a native atrial septum. The solid wall tube prevents tissue overgrowth across the atrial septum, and can have minimal exposure of the device in the left atrium to minimize stroke and other risks. In some examples, the anchoring assembly is configured to be self-transitionable from a delivery state to a deployed state.

### CROSS-REFERENCE TO RELATED APPLICATIONS

This Non-Provisional Patent Application claims the benefit of the filing date of U.S. Provisional Patent Application No. 63/248,866, filed Sep. 27, 2021, entitled “INTERATRIAL SHUNTING DEVICES, SYSTEMS AND METHODS”, the entire teachings of which are incorporated herein by reference.

### BACKGROUND

The present disclosure relates to systems, devices and methods for treating heart failure. More particularly, it relates to interatrial shunting systems, devices and methods for reducing elevated blood pressure in a heart chamber, for example by establishing a pressure relieving shunt across at an interatrial septum to reduce left atrial pressure.

Heart failure is a condition where the heart cannot pump blood properly, for example because the heart has become weak. The heart muscles can become weak due to a variety of different causes, but some of the most common include coronary heart disease and high blood pressure. As a result of this weakening, blood flows backwards and builds up in the left side of the heart, causing increased pressure in the heart. One sub-type of heart failure is characterized by the heart being able to pump blood well but not relax, leading to increased pressure in the heart. Heart failure with preserved ejection fraction (HFpEF), also known as diastolic heart failure, occurs when the lower left ventricle is not able fill properly with blood during the diastolic phase. Over time, this increased filling causes blood to build up inside the left atrium. The adverse result of these and other heart failure conditions leads to elevated pressure in the left atrium.

Various treatments for heart failure have been suggested with varying degrees of success, including pharmacological, implanted assist devices, and surgical treatments. Some symptoms of certain types of heart failure (e.g., diastolic heart failure) can be improved or addressed by relieving pressure from one chamber of the heart to another.

### SUMMARY

The inventors of the present disclosure recognized that a need exists for improved systems, devices and methods for treating heart failure.

Some aspects of the present disclosure relate to an interatrial shunting device including a tube and an anchoring assembly. The tube defines a first end opposite a second end, and a tube wall extending to and between the first and second ends. The tube wall is a solid body and defines a lumen of the tube, with the lumen being open at the first and second ends. The anchoring assembly is carried by the tube and is configured to secure the interatrial shunting device to a native atrial septum. With this construction, the solid wall tube prevents tissue overgrowth across the atrial septum, and can have minimal exposure of the device in the left atrium to minimize stroke and other risks. In some examples, the anchoring assembly is configured to be self-transitionable from a delivery state to a deployed state. In the deployed state, the anchoring assembly readily engages the atrial septum, whereas the delivery state has a reduced radial footprint conducive to catheter-based delivery.

Other aspects of the present disclosure relate to a system for treating a heart of a patient, the system including an interatrial shunting device and a delivery device. The interatrial shunting device includes a tube and an anchoring assembly. The tube defines a first end opposite a second end, and a tube wall extending to and between the first and second ends. The tube wall is a solid body and defines a lumen of the tube, with the lumen being open at the first and second ends. The anchoring assembly is carried by the tube and is configured to secure the interatrial shunting device to a native atrial septum. The delivery device is configured to retain the interatrial shunting device in a delivery state for delivery to a native atrial septum and to release the interatrial shunting device for implant at an opening in the native atrial septum in a deployed state. In some embodiments, the delivery device includes an engagement unit slidably disposed within an outer sheath. The engagement unit is configured for temporary connection to structural features of the anchoring assembly, for example one or more arms extending from the tube.

Yet other aspects of the present disclosure relate to a method of treating heart failure. The method includes forming a hole in an atrial septum of the heart. An interatrial shunting device is implanted at the hole. In this regard, the step of implanting includes positioning a tube of the interatrial shunting device within the hole. The tube defining a first end opposite a second end, and a tube wall extending to and between the first and second ends. The tube wall is a solid body and defines a lumen of the tube, with the lumen being open at the first and second ends. The lumen provides a long term fluid passage across the atrial septum. With these and related methods, tissue ingrowth across the lumen following implant is minimized.

### BRIEF DESCRIPTION OF THE DRAWINGS

FIG. 1A is a perspective view of an interatrial shunting device in accordance with principles of the present disclosure;

FIG. 1B is a top plan view of the interatrial shunting device of FIG. 1A;

FIG. 1C is a side view of the interatrial shunting device of FIG. 1A;

FIG. 2A is a perspective view of the interatrial shunting device of FIG. 1A arranged in a delivery state;

FIG. 2B is a side view of the arrangement of FIG. 2A;

FIG. 3A is an enlarged, cross-sectional view of a portion of the interatrial shunting device of FIG. 1B, taken along the line 3A-3A;

FIG. 3B is an enlarged, cross-sectional view of a portion of the interatrial shunting device of FIG. 1C, taken along the line 3B-3B;

FIG. 4 is a simplified side view of a delivery device in accordance with principles of the present disclosure and useful, for example, in delivering an interatrial shunting device to a target site;

FIG. 5 is an exploded, perspective view of portions of a delivery device useful with the delivery device of FIG. 4, including portions of an outer sheath assembly and portions of an inner shaft assembly;

FIG. 6 is an enlarged, cross-sectional view of a portion of the outer sheath assembly of FIG. 5;

FIG. 7A is a perspective view of an engagement unit useful with the inner shaft assembly of FIG. 5;

FIG. 7B is a side view of the engagement unit of FIG. 7A;

FIG. 7C is an end view of the engagement unit of FIG. 7A;

FIGS. 8A and 8B are perspective views illustrating complimentary connection features provided with the engagement unit of FIG. 7A and the interatrial shunting device of FIG. 1A;

FIG. 9 is a perspective view illustrating an initial stage of connection between the engagement unit of FIG. 7A and the interatrial shunting device of FIG. 1A;

FIG. 10 is a perspective view of an alternative engagement unit useful with the delivery devices of the present disclosure;

FIG. 11 is a perspective view of portions of the outer sheath assembly and the inner shaft assembly of FIG. 5A connected to the interatrial shunting device of FIG. 1A;

FIG. 12 is an enlarged, cross-sectional view of a portion of a treatment system in accordance with principles of the present disclosure, including the interatrial shunting device of FIG. 1A loaded to the delivery device of FIG. 5 in a delivery arrangement;

FIG. 13 illustrates anatomy of a human heart, including an atrial septum with which devices and methods of the present disclosure are useful;

FIG. 14A is a simplified side view of an initial stage of methods of the present disclosure, including a delivery device loaded with an interatrial shunting device arranged relative to a atrial septum hole;

FIG. 14B is a cross-sectional view of the initial stage of FIG. 14A;

FIG. 15A is a simplified side view of illustrating a later stage of methods of the present disclosure subsequent to the stage of FIG. 14A, including partial deployment of the interatrial shunting device;

FIG. 15B is a cross-section view of the later stage of FIG. 15A;

FIGS. 16 and 17 illustrate later stages of methods of the present disclosure subsequent to the stage of FIG. 15A, including implanting of the interatrial shunting device; and

FIGS. 18A and 18B illustrate final implant of the interatrial shunting device of FIG. 1A to an atrial septum.

### DETAILED DESCRIPTION

Aspects of the disclosure are directed to an interatrial shunting device, delivery devices, and methods of use.

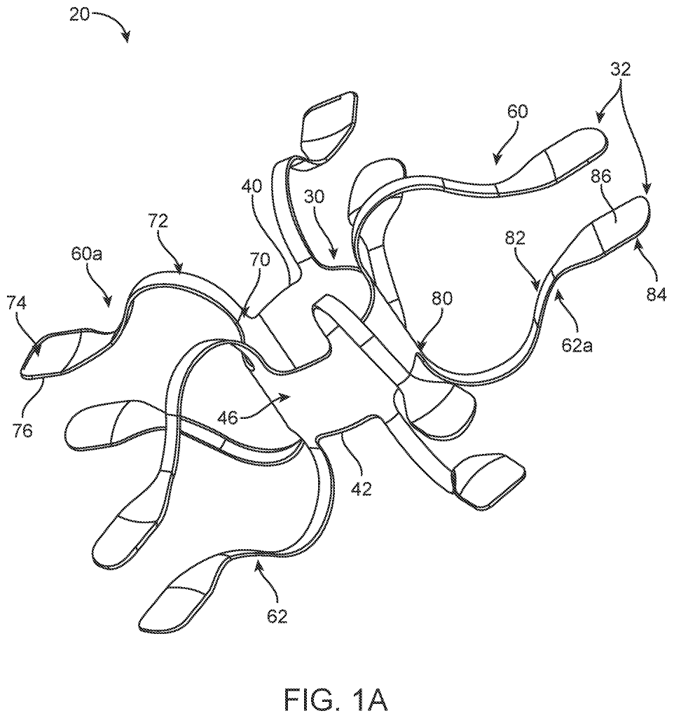

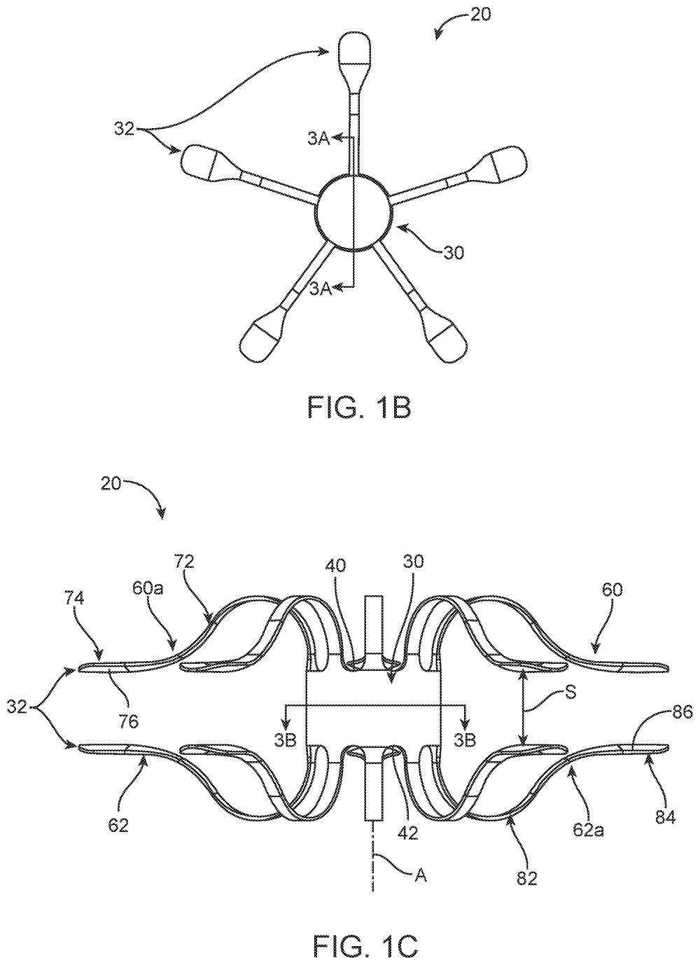

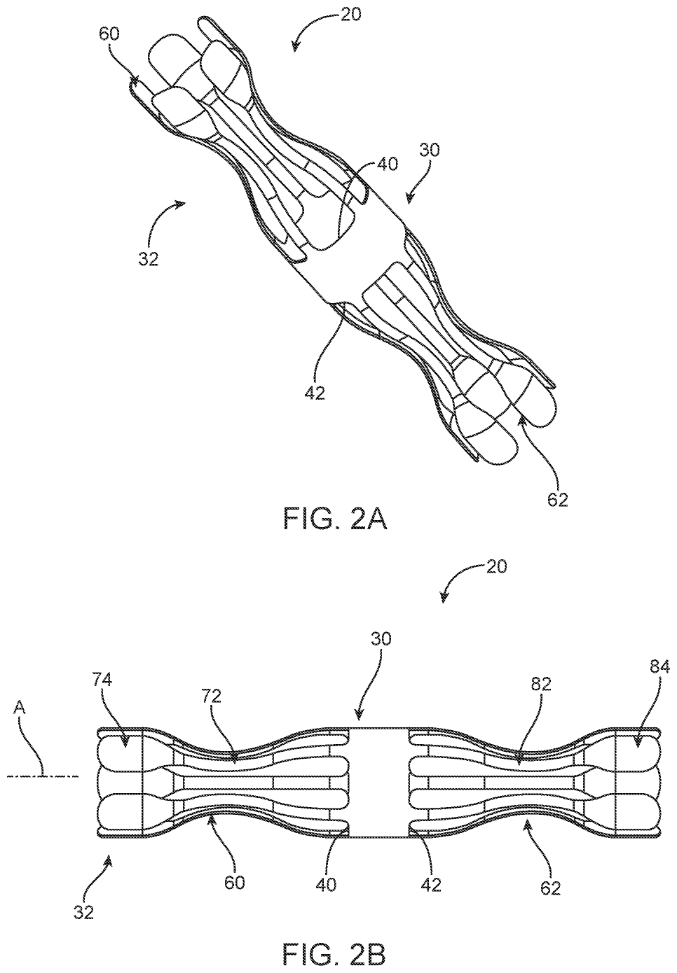

One embodiment of an interatrial shunting device 20 in accordance with principles of the present disclosure is shown in FIGS. 1A-1C. The interatrial shunting device 20 includes or defines a tube 30 and an anchoring assembly 32 (referenced generally). As described in greater detail below, the tube 30 is configured for placement within a hole formed through a tissue wall of a patient (e.g., an interatrial septum), establishing and maintaining an unobstructed fluid pathway across the tissue wall (e.g., in some embodiments, the interatrial shunting device 20 is characterized by the absence of materials, bodies, mechanism, etc., within the pathway defined by the tube 30). The anchoring assembly 32 is carried by or connected to the tube 30, and is configured to promote anchoring of the interatrial shunting device 20 with native tissue in a final implant location. In some embodiments, the interatrial shunting device 20 is configured for percutaneous delivery to a target site (e.g., a hole formed through an interatrial septum) via a catheter-based device or the like; with these and related embodiments, the anchoring assembly 32 can be configured to readily assume a format conducive to catheter-based delivery as described below. With this in mind and as a point of reference, FIGS. 1A-1C depict the interatrial shunting device 20 with the anchoring assembly 32 in a normal or deployed state, whereas FIGS. 2A and 2B illustrate one example of a delivery state. In some examples, an external force can direct or deflect the anchoring assembly 32 to the delivery state of FIGS. 2A and 2B. The anchoring assembly 32 is optionally configured to self-revert or self-transition to the deployed state of FIGS. 1A-1C upon removal of the external force.

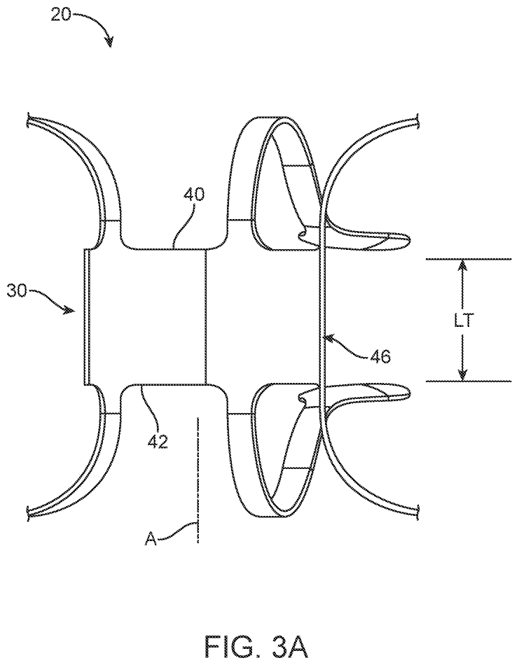

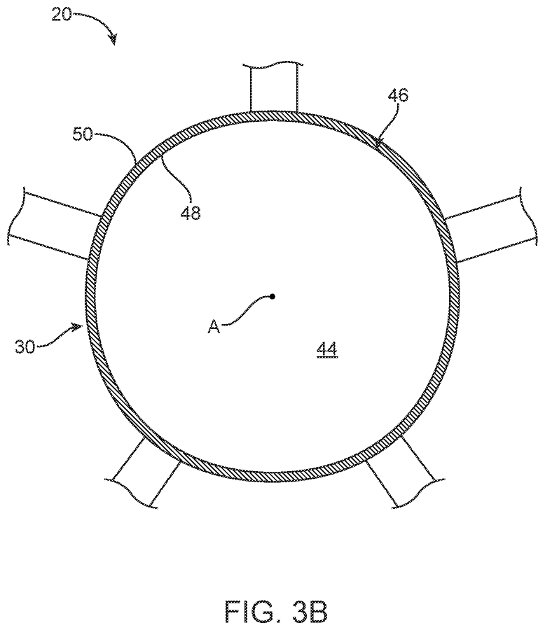

With additional reference to FIGS. 3A and 3B, the tube 30 defines a first end 40 opposite a second end 42, and a lumen 44. The lumen 44 extends along a longitudinal axis A of the tube 30, and is open at each of the first and second ends 40, 42. A structure of the tube 30 is generated by a tube wall 46 defining an inner surface 48 opposite an outer surface 50. The tube 30 is formed of a biocompatible material, and is configured to minimize or prevent tissue ingrowth across a thickness of, and/or to an inside of, the tube wall 46. In some embodiments, these properties or features of the tube 30 can be characterized by the outer surface 50 being in direct contact with living human tissue (e.g., interatrial septal tissue) and no tissue ingrowth occurs across a thickness of, and/or to an inside of, the tube wall 46 (e.g., from the outer surface 50 to the inner surface 48 and into the lumen 44) for at least 180 days and can, in some embodiments, be considered a permanent attribute of the tube 30 following implant.

The tube 30 can incorporate various features that serve to minimize or prevent tissue ingrowth. For example, the tube wall 46 is formed as a solid body as reflected, for example, in FIGS. 3A and 3B. The solid nature of the tube wall 46 can include the tube wall 46 being an integral, homogenous body and exhibiting a porosity (if any) across a thickness of the tube wall 46 that is less than the pore size necessary for human tissue to progressively grow. That is to say, while material of the tube wall 46 may have a naturally-occurring porosity or interstitial spacing, human tissue cannot grow across the tube wall 46. For example, in some embodiments, the tube 30 is formed of a nickel titanium allow (e.g., Nitinol®), although other biologically compatible materials (e.g., metals or metal alloys) are acceptable. The solid construction of the tube wall 46 is or exists, in some embodiments, across an entire length of the tube 30 from the first end 40 to the second end 42. For example, the tube wall 46 can be described as being solid in transverse cross-section (e.g., the cross-sectional plane of FIG. 3B) across the entire length of the tube 30 from the first end 40 to the second end 42. In addition or alternatively, in some embodiments a blood compatible coating is applied to at least the tube 30. Blood compatible coatings of the present disclosure can assume various forms, and in some embodiments includes or consists of a phosphoryl-choline biomolecule that is covalently attached to a surface of the Nitinol® (or similar material) tube 30 via intermediate silane molecules, for example Shield Technology™ available from Medtronic, Inc. In some non-limiting examples, a Nitinol® (or similar) substrate useful to form the tube 30 (and optionally an entirety of the interatrial shunt device 20) can be electropolished. To generate surface hydroxyl groups, the substrate can be subjected to one of several surface treatments including air plasma, oxygen gas plasma, base (NaOH), acid, etc. Next, the substrate can be immersed in an appropriate solution, such as 3-glycidyloxypropyltrimethoxysilane (GPTS) solution. The pretreated substrate reacts with a phosphorylcholine (PC) polymer, for example, 2-methacryloyoxyethil phosphorylcholine. Other techniques and/or materials can be employed to generate a blood compatible coating on at least a surface of the tube 30, for example blood compatible or anti-thrombogenic coatings or techniques described in U.S. Pat. Nos. 8,709,465, 9,668,890, or 9,545,301, the entire teachings of each of which are incorporated herein by reference.

In some embodiments, the tube wall 46, and thus the tube 30, is not stent and does not have a stent-like structure (e.g., a series of wires or struts welded to one another, a metal tube cut to form a series of interconnected struts, etc.). In some embodiments, the tube 30 is configured (e.g., exhibits sufficient hoop strength) to not overtly deflect or change shape in response to a radially compressive force (e.g., a diameter of tube 30 is fixed and will not change in the presence of a radially compressive force otherwise used to effect deflection of the anchoring device 32 as described below).

In some embodiments, dimensions of the tube 30 are selected to establish the lumen 44 of sufficient size (e.g., cross-sectional area) for adequate interatrial shunting while minimizing possible impediments to other expected native anatomy and heart functioning. For example, in some embodiments, the tube 30 has an outer diameter in the range of 10-50 French (0.13-0.66 inch; 3.33-16.67 millimeters (mm)), alternatively in the range of 15-24 French (0.2-0.31 inch; 5-8 mm), alternatively on the order of 19 French (0.25 inch; 6.33 mm). In some embodiments, a length LT of the tube 30 (i.e., linear distance from the first end 40 to the second end 42) is in the range of 4-80 mm, for example a length on the order of 5 mm. In some embodiments, the length of the tube 30 is selected to approximate (e.g., be slightly greater than) an expected thickness of the atrial septum to which the interatrial shunting device 20 will be implanted. For example, in some non-limiting embodiments, the interatrial shunting device 20 is intended to be implanted at the interatrial septum separating the left and right atriums; with these and related embodiments, the tube 30 will extend across a thickness of the interatrial septum and has a length only slightly greater (e.g., 4-5 mm greater) than the expected thickness of the interatrial septum. With these and related embodiments, exposure of the so-dimensioned tube 30 in the left atrium is beneficially minimized. Other dimensions (e.g., diameter, length, etc.) are also envisioned.

Returning to FIGS. 1A-1C, 2A, and 2B, the anchoring assembly 32 can assume various forms or formats conducive to retaining the interatrial shunting device 20 at a target site, with the tube 30 being implanted and held within a hole formed in an interatrial septum. In some embodiments, the anchoring assembly 32 is configured to readily assume a format conducive to catheter-based delivery and self-deploy or self-transition to the deployed state of FIGS. 1A-1C when released from the catheter (or other force applied to the anchoring assembly 32 during delivery). In one non-limiting example, the anchoring assembly 32 includes one or more first arms 60 and one or more second arms 62. Each of the first arms 60 extends from the first end 40 of the tube 30, and each of the second arms 62 extends from the second end 42. In some embodiments, the anchoring assembly 32 includes the same number of the first and second arms 60, 62. With the non-limiting example of FIGS. 1A-1C, five of the first arms 60 and five of the second arms 62 are provided, although any other number, either greater or lesser, is acceptable, and the same number of first and second arms 60, 62 is not required. Moreover, while in some embodiments each of the first arms 60 is circumferentially aligned with a corresponding one of the second arms 62 (best reflected by FIG. 1C), in other embodiments, one or more of the first arms 60 can be circumferentially offset relative to all of the second arms 62 (and vice-versa).

The first arms 60 can, in some embodiments, have an identical shape and construction.

Thus, the following description of the first arm labeled at 60a in FIGS. 1A and 1C can apply equally to the remaining first arms 60. The first arm 60a forms or defines a base 70 at the point of connection or attachment to the tube 30. In some embodiments, the base 70 extends directly from the first end 40, although in other examples, the base 70 can be located apart from the first end 40. Regardless, the first arm 60a defines an intermediate section 72 in extension from the base 70, and terminates at a head 74 opposite the base 70. The head 74 has, in some embodiments, an enlarged size or surface area as compared to the intermediate section 72, forming a contact face 76 (referenced generally) selected to promote atraumatic interface with a tissue wall (e.g., interatrial septum). The intermediate section 72 defines a curve in extension from the base 70 to the head 74. A curvature of the intermediate section 72 can define a bend angle on the order of 90 degrees in some embodiments such that the contact face 76 generally faces the second end 42 and/or is generally perpendicular to the longitudinal axis A in the deployed state. Other shapes and orientations are also envisioned. Regardless, a material and construction of the first arm 60a has shape memory properties such that the first arm 60a self-assumes the geometry of FIGS. 1A-1C (e.g., the first arm 60a can be formed of a shape memory metal alloy such as Nitinol®). The first arms 60 can be forced to deflect from the deployed state (e.g., deflected to the delivery state of FIGS. 2A and 2B), and will self-revert back to or towards the shape and orientation of deployed state (FIGS. 1A-1C) for reasons made clear below.

The second arms 62 can, in some embodiments, have an identical shape and construction. Moreover, the second arms 62 can have the shape and constructions described above with respect to the first arms 60. Thus, the following description of the second arm labeled at 62a in FIGS. 1A and 1C can apply equally to the remaining second arms 62. The second arm 62a forms or defines a base 80 at the point of connection or attachment to the tube 30. In some embodiments, the base 80 extends directly from the second end 42. The second arm 62a defines an intermediate section 82 in extension from the base 80, and terminates at a head 84 opposite the base 80. The head 84 forms an enlarged surface area contact face 86. The intermediate section 82 defines a curve in extension from the base 80 to the head 84, for example a bend angle on the order of 90 degrees such that the contact face 86 generally faces the first end 40 and/or is generally perpendicular to the longitudinal axis A in the deployed state. The second arm 62a has shape memory properties such that the second arm 62a self-assumes the geometry of FIGS. 1A-1C. The second arms 62 can be forced to deflect from the deployed state (e.g., deflected to the delivery state of FIGS. 2A and 2B), and will self-revert back to or towards the shape and orientation of deployed state (FIGS. 1A-1C) for reasons made clear below.

As best shown in FIG. 1C, in the deployed state, the arms 60, 62 maintain the corresponding contact faces 76, 86 at a longitudinal spacing S. In some embodiments, the longitudinal spacing S is selected to be slightly less than an expected thickness of the interatrial septum to which the interatrial shunting device 20 will be implanted. With this but one example, then, the anchoring device 32 is configured to capture a thickness of the interatrial between the contact faces 76, 86. In some embodiments, the longitudinal spacing S is in the range of 2-10 mm, for example approximately 3.5 mm, although other dimension are also acceptable. As a point of reference, under circumstances where the structure interposed between the contact faces 76, 86 has a thickness greater than the longitudinal spacing S normally established by the arms 60, 62, one or more of the intermediate sections 72, 82 readily deflects to accommodate the thickness; the shape memory attribute of the arms 60, 62, however, continues to force or drive the heads 74, 84 toward one another (toward the normal longitudinal spacing S), serving to robustly engage the structure between the contact faces 76, 86.

As mentioned above, the arms 60, 62 are, in some embodiments, configured to be forced or deflected away from the deployed state, and then self-return or self-revert back to the deployed state. For example, the arms 60, 62 can be deflected to the delivery state of FIGS. 2A and 2B. As a point of reference, external forces acting upon the arms 60, 62 to effect the delivery state are not represented in FIGS. 2A and 2B for ease of understanding. In the delivery state, and as best seen in FIG. 2B, the first arms 60 have been deflected (as compared to the deployed state) such that the corresponding intermediate section 72 extends primarily in a first direction relative to the longitudinal axis A, locating the corresponding head 74 away from the first end 40 (e.g., the head 74 of each of the first arms 60 is longitudinally spaced from the first end 40 of the tube 30 in a direction opposite the second end 42). The second arms 62 have been deflected (as compared to the deployed state) such that the corresponding intermediate section 82 extends primarily in a second direction relative to the longitudinal axis A that is opposite of the first direction, locating the corresponding head 84 away from the second end 42 (e.g., the head 84 of each of the first arms 62 is longitudinally spaced from the second end 42 of the tube 30 in a direction opposite the first end 40). With these and other constructions, a footprint of the anchoring assembly 32 in a plane transverse to the longitudinal axis A in the delivery state is less than the footprint in the deployed state and is conducive to placement within a catheter or other tubular body. However, a shape, geometry and dimensions of the tube 30 are substantially identical (i.e., within 5 percent of truly identical) in the deployed and delivery states.

In some embodiments, the anchoring assembly 32 (i.e., the first and second arms 60, 62) is integrally formed with the tube 30. For example, an entirety of the interatrial shunting device 20 can be an integral, homogenous body formed from a biocompatible material such a Nitinol® or the like. In other embodiments, one or more or all components of the anchoring assembly 32 can be separated formed and subsequently assembled to the tube 30. Moreover, anchoring assemblies useful with the interatrial shunting devices of the present disclosure can assume a wide variety of other designs that may or may not be directly implicated by the views. Virtually any anchoring format useful for maintaining the tube 30 relative to an interatrial septum hole can be employed.

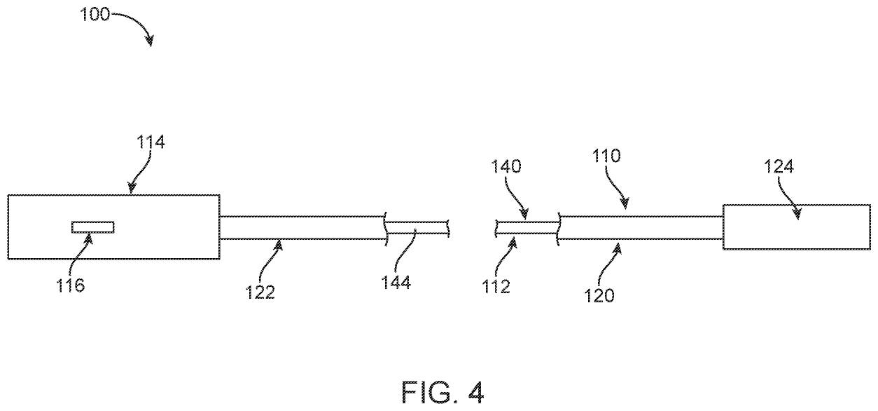

Some aspects of the present disclosure relate to devices or tools for delivering the interatrial shunting device, such as the interatrial shunting device 20, in a percutaneous fashion to an interatrial septum target site, for example a catheter-based or catheter-type device. In general terms, the delivery devices of the present disclosure are configured to retain the interatrial shunting device in a delivery state for delivery to a native atrial septum and to release the interatrial shunting device for implant at an opening in the native atrial septum in a deployed state. With this in mind, one non-limiting example of a delivery device 100 in accordance with principles of the present disclosure and useful, for example, in delivering the interatrial shunting device 20 (FIG. 1A) is schematically reflected by FIG. 4. The delivery device 100 includes an outer sheath assembly 110, an inner shaft assembly 112, and a handle assembly 114. Details on the various components are provided below. In general terms, the inner shaft assembly 112 is configured to selectively engage a segment of an interatrial shunting device (e.g., the interatrial shunting device 20 of FIG. 1A), and is slidably disposed within the outer sheath assembly 110. The outer sheath assembly 110 incorporates features (e.g., a capsule) appropriate for containing the interatrial shunting device in a delivery state. The handle assembly 114 connected to the inner shaft assembly 112, and provides features (e.g., an actuator 116) that facilitate user-prompted, sliding articulation of the inner shaft assembly 112 relative to the outer sheath assembly 110 (and/or vice-versa). With this construction, an interatrial shunting device can be loaded to the delivery device 100 and maintained in a delivery state for delivery to a target site. The handle assembly 114 is manipulated by the user to direct the loaded interatrial shunting device to a target site, followed by operation of the handle assembly 114 to effect implant of the interatrial shunting device at the target site. For example, the actuator 116 can be operated by the user to progressively advance the inner shaft assembly 112 relative to the outer sheath assembly 110, causing the interatrial shunting device to deploy. The delivery device 100 can incorporate various features by which a user can effect release of the interatrial shunting device therefrom. As a point of reference, the handle assembly 114 can assume various constructions and include various features that facilitate user handling and operation.

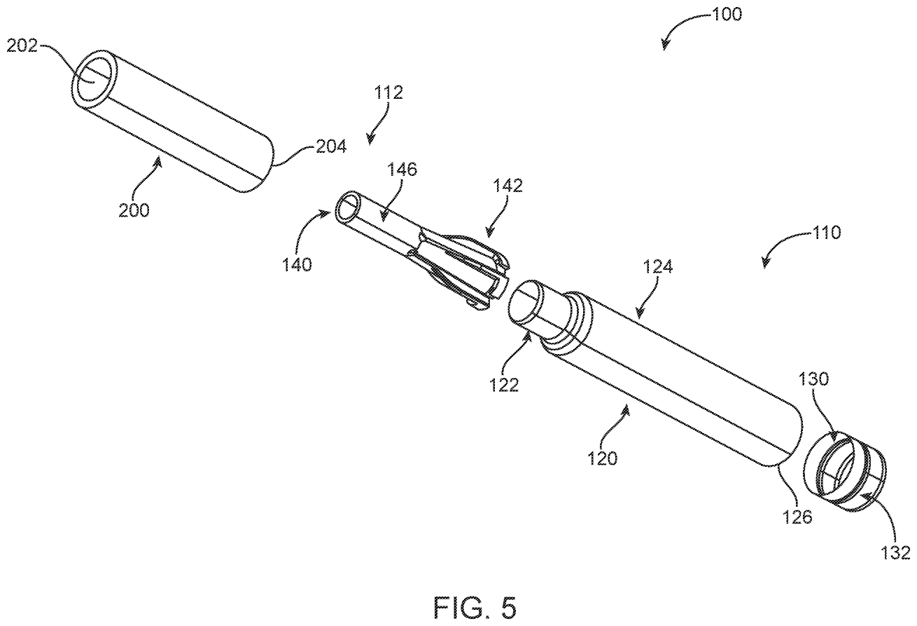

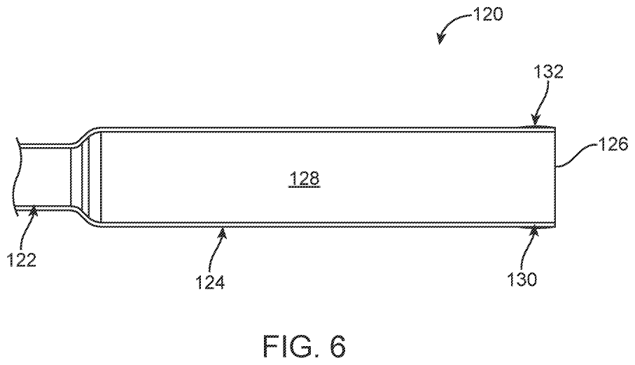

The outer sheath assembly 110 and the inner shaft assembly 112 can assume a variety of forms; portions of some non-limiting examples of the outer sheath assembly 110 and the inner shaft assembly 112 are shown in greater detail in FIG. 5. The outer sheath assembly 110 includes an outer sheath 120 defining a proximal region 122 and a capsule 124. As further shown in FIG. 4, the proximal region 122 extends proximally from the capsule 124 (only a portion of the proximal region 122 is illustrated in FIG. 5) and is connected the handle assembly 114. The proximal region 122 can have any construction conventionally employed for catheter-based, percutaneous cardiac procedures (e.g., a polymer tube, reinforced polymer tube, etc.). The capsule 124 can have a more stiffened construction (as compared to a stiffness of the proximal region 122) that exhibits sufficient radial or circumferential rigidity to overtly resist expected expansive forces of the interatrial shunting device (not shown) in a delivery state. The capsule 124 is constructed, in some embodiments, to compressively retain the interatrial shunting device (e.g., the interatrial shunting device 20 (FIG. 1A)) when loaded within the capsule 124, and the proximal region 122 serves to connect the capsule 124 with the handle assembly 114. The capsule 124 extends distally from the proximal region 122 and terminates at a distal end 126. As best shown in FIG. 6, the capsule 124 defines an interior passage 128 that, in some embodiments, has a diameter that is greater than an inner diameter of the proximal region 122 (e.g., FIG. 6 reflects that the inner diameter of the outer sheath 120 expands in the distal direction at a transition to the capsule 124), with the enlarged diameter interior passage 128 being open at the distal end 126 and having a length approximating a length of the interatrial shunting device in the delivery state as describe in greater detail below.

With continued reference between FIGS. 5 and 6, the outer sheath assembly 110 can include one or more additional, optional components. For example, a marker band 130 (e.g., a radiopaque marker band) can be formed or assembled to the capsule 124 at or in close proximity to the distal end 126. Where provided, the marker band 130 can assist a clinician in locating the distal end 126 during an implant procedure. An outer reinforcement layer 132 can be applied over at least a distal segment of the capsule 124 in some optional embodiments. Where provided, the outer reinforcement layer 132 can cover the marker band 130 in an atraumatic manner and/or can provide additional hoop strength to the capsule 124 at a region where expansive forces created by a loaded interatrial shunting device (not shown) are expected to be applied.

With specific reference to FIGS. 4 and 5, the inner shaft assembly 112 includes an inner shaft 140 (referenced generally in FIG. 5) attached to or carrying an engagement unit 142. The inner shaft 140 can be a tubular or solid body, and includes or defines a proximal section 144. The proximal section 144 extends proximally from the engagement unit 142 and is connected the handle assembly 114.

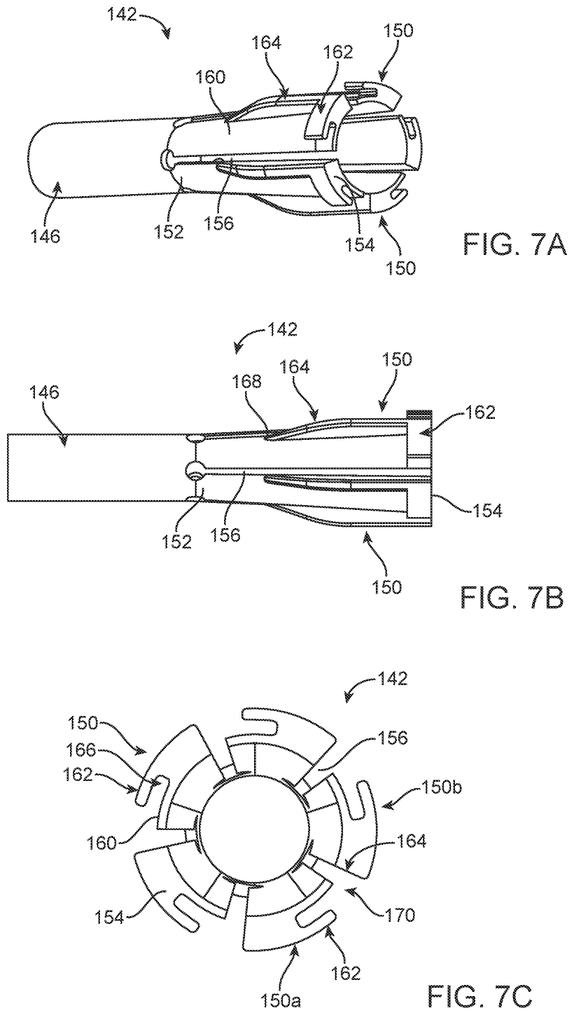

The engagement unit 142 can be integrally formed with the proximal section 144. Alternatively, the engagement unit 142 can include or define a connector body 146 configured for attachment to the proximal section 144. Regardless, the engagement unit 142 incorporates various features configured to facilitate selective engagement with corresponding features of an interatrial shunting device, for example features of the interatrial shunting device 20 (FIG. 1A) described above. One non-limiting example of the engagement unit 142 is shown in greater detail in FIGS. 7A-7C, and includes the connector body 146 and a plurality of fingers 150. Each of the fingers 150 extends distally from a base end 152 at the connector body 146 to a leading end 154. The fingers 150 are arranged in a symmetric, circumferential pattern. Circumferentially adjacent ones of the fingers 150 are separated from one another by a longitudinal gap 156. With this construction, then, the fingers 150 can each deflect relative to one another, pivoting at the corresponding base end 152.

The fingers 150 can have an identical construction, each defining a floor surface 160, a tab 162, and a guide shoulder 164 (labeled for one of the fingers 150 in FIG. 7A). The tab 162 is formed at the leading end 154, and combines with the floor surface 160 to define a capture slot 166. The guide shoulder 164 is formed as a radially outward projection from the floor surface 160, extending from the tab 162 to a leading end 168. Relative to a pair of circumferentially adjacent ones of the fingers 150 (e.g., the fingers labeled as 150a, 150b labeled in FIG. 7C), a clearance zone 170 is defined between a terminal edge of the tab 162 of the first finger 150a and the guide shoulder 164 of the second finger 150b.

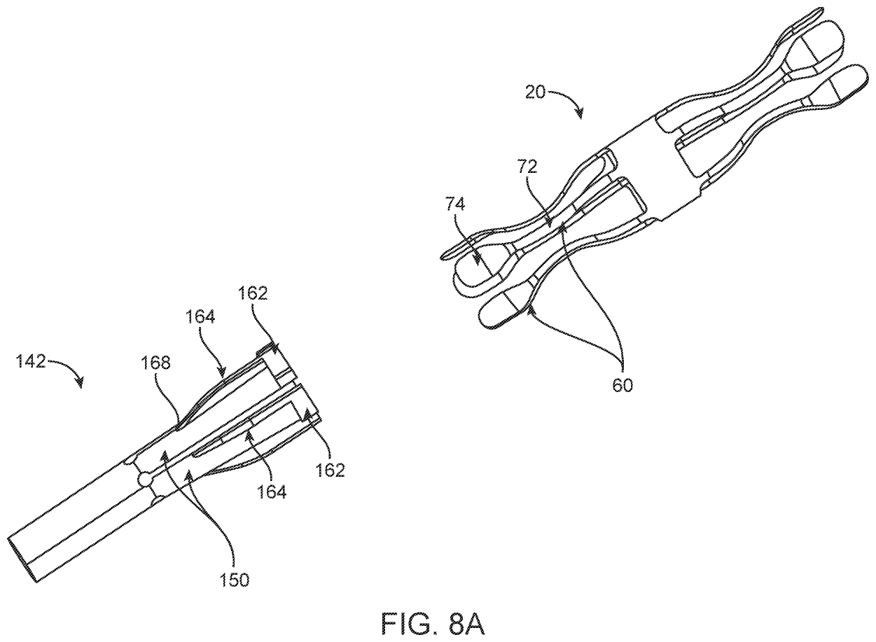

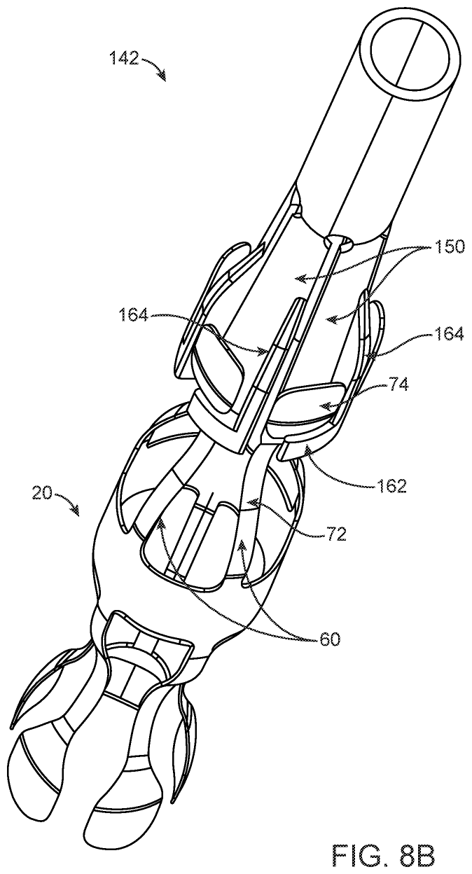

With additional reference to FIGS. 8A and 8B, various geometries and other attributes of the fingers 150 correspond with features of the interatrial shunting device 20. As a point of reference, in the views of FIGS. 8A and 8B, the interatrial shunting device 20 and the engagement unit 142 are illustrated in isolation with the interatrial shunting device 20 arranged in the delivery state. It will be understood that the external force(s) causing the interatrial shunting device 20 to transition from the deployed state (FIG. 1A) to the delivery state are not specifically identified for ease of illustration. In the arrangement of FIG. 8A, the engagement unit 142 is poised for connected to the interatrial shunting device 20, whereas FIG. 8B illustrates final connection between the two components 20, 142.

With this in mind, the number of fingers 150 provided with the engagement unit 142 corresponds with the number of first arms 60 provided with the interatrial shunting device 20 (e.g., five in some non-limiting examples). A height of the slot 164 (best seen in FIG. 7C) provided with each of the fingers 150 is slightly greater than (or otherwise corresponds with) a thickness of the intermediate section 72 of each of the first arms 60. Further, a size of the circumferential spacing between adjacent ones of the tabs 162 is slightly greater than (or otherwise corresponds with) a width of the intermediate section 72 of each of the first arms 60. A circumferential spacing between adjacent ones of the guide shoulders 164 is slightly greater than (or otherwise correspond with) a width of the head 74 of each of the first arms 60. Finally, a longitudinal length of the guide shoulder 164 (i.e., longitudinal distance from the tab 162 to the leading end 168) is less than the longitudinal length of the intermediate section 72 of each of the first arms 60.

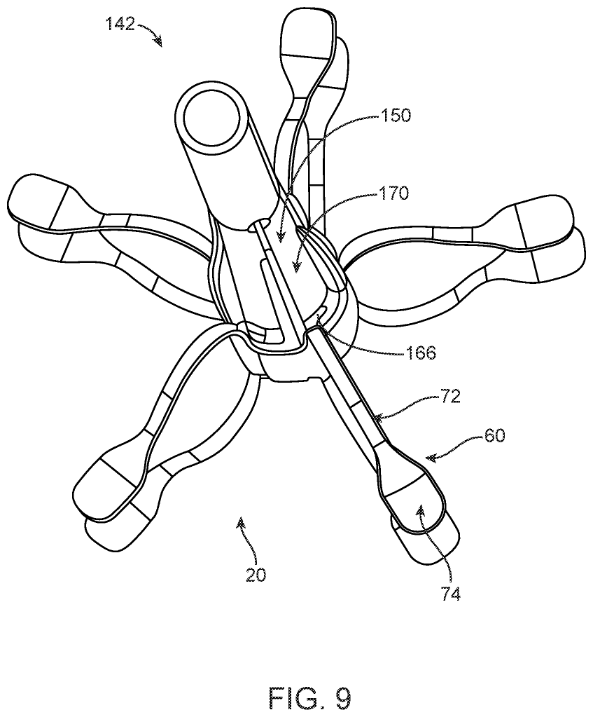

With this construction, the interatrial shunting device 20 can be connected to the engagement unit 142 by circumferentially arranging the engagement unit 142 relative to the interatrial shunting device 20 (and/or vice-versa) such that respective ones of the clearance zones 170 (FIG. 7C) are aligned with the intermediate section 72 of a corresponding one of the first arms 60 as represented, for example, by FIG. 9 (it being understood that in the view of FIG. 9, the interatrial shunting device 20 is shown in the deployed state). The interatrial shunting device 20 and the engagement unit 142 are then articulated relative to one another (e.g., rotated), bringing the intermediate section 72 of each of the first arms 60 into a corresponding one of the slots 166. Thus, the intermediate section 72 of each of the first arms 60 is captured by a corresponding one of the fingers 150. From this arrangement, the interatrial shunting device 20 is moved longitudinally away from the engagement unit 142 (and/or vice-versa), with the intermediate section 72 of each of the first arms 60 sliding within the corresponding slot 166. With this sliding motion, the tab 162 (best seen in FIGS. 7A-7C) applies a force onto the corresponding first arm 60, causing the corresponding intermediate section 72 to deflect toward the delivery state. Further, the head 74 of each of the first arms 60 will eventually be directed into the circumferential spacing between the guide shoulders 164. In the final arrangement of FIG. 8B, the head 74 of each of the first arms 60 is immediately adjacent the tab 162 of the corresponding finger 150 and is captured between the guide shoulders 164 of the corresponding pair of fingers 150. Thus, interface between the head 74 and the guide shoulder(s) 164 ensures that the intermediate section 72 remains in the corresponding slot 166. The shape memory bias of each of the first arms 60 toward the deployed state is resisted by the corresponding tab 162, thereby radially restraining or locking the first arms 60 in the delivery state. The interatrial shunting device 20 can be released from the engagement unit 142 by reversing the steps.

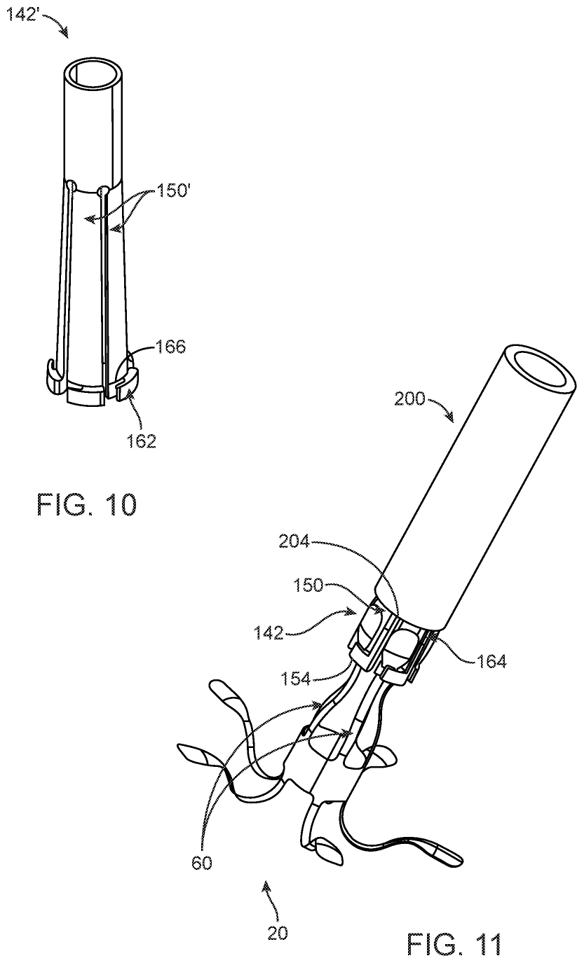

The engagement unit 142 as described above is but one acceptable configuration envisioned by the present disclosure. One non-limiting example of another engagement unit 142′ is shown in FIG. 10. The engagement unit 142′ can be highly akin to the engagement unit 142 (FIG. 7A), including a plurality of deflectable fingers 150′ each providing the tab 162 and capture slot 166 as described above. In yet other embodiments, the engagement units of the present disclosure can have other constructions or configurations appropriate for temporary assembly or connection to an interatrial shunting device that may or may not be directly implicated by the views.

Returning to FIG. 5, the delivery device 100 can optionally include one or more additional components, for example a lock tube 200. Where provided, the lock tube 200 is configured to interface with at least the engagement unit 142 in dictating a diameter collectively defined by the fingers 150. In particular, the lock tube 200 can be a reinforced tubular body, defining a lumen 202 (referenced generally) open to a distal end 204. It will be recalled that the fingers 150 can each deflect or pivot relative to the connection body 146. Thus, in the presence of a radially expansive force (e.g., applied by the interatrial shunting device 20 loaded to the engagement unit 142 as described above and shown, for example, in FIG. 8B), the fingers 150 may be caused to deflect radially outwardly, with the leading ends 154 collectively defining an increased diameter. The lock tube 200 can be employed to resist this expansion or cause the so-arranged figures 150 to deflect, forcing the leadings ends 154 radially inwardly, thus decreasing the diameter collectively defined by the leading ends 154. In particular, and with additional reference to FIG. 11, the lock tube 200 can be inserted over the connection body 146 (hidden in FIG. 11 but visible, for example, in FIG. 5) and distally advanced toward the fingers 150. With continued distal advancement of the distal end 204 of the lock tube 200 relative to the engagement unit 142 (and/or proximal retraction of the engagement unit 142 relative to the distal end 204), the guide shoulders 164 eventually enter the lumen 202 and contact an interior surface of the lock tube 200. With this arrangement, the lock tube 200 exerts a compressive force onto the fingers 150 (via interface with the corresponding guide shoulders 164), causing the leading ends 154 to collectively move radially inwardly. Thus, where the interatrial shunting device 20 is mounted to the engagement unit 142 with, for example, the first arms 60 exerting a radially outward or expansive force onto the fingers 150, presence of the lock tube 200 can assist in opposing the expansive force, maintaining the leading ends 154, and thus the first arms 60 as secured thereto, at a reduced collective diameter than might otherwise occur absent the lock tube 200. Alternatively or in addition, the lock tube 200 can be distally advanced beyond the location implicated by FIG. 11 and over at least a portion of the first arms 60 to effect further securement of the interatrial shunting device 20 with the engagement unit 142. With these and related embodiments, the lock tube 200 can be sized and shaped to be received within the outer sheath 120, including the proximal region 122 and the capsule 124. The lock tube 200 extends to the handle assembly 114 and is connected to an actuator of the handle assembly 114 that affords a user the ability to distally advance and proximally retract the lock tube 200. The lock tube 200 can be employed to hold the fingers 150, and thus the first arms 60, while the capsule 124 is being retracted as described below. This may provide more flexibility by moving the outer sheath assembly 110 away from the interatrial shunting device 20 during final steps of the deployment procedures described below. The lock tube 200 can assume a wide variety of other forms, and in other embodiments is omitted. For example, the capsule 124 can be configured (e.g., exhibit sufficient hoop strength or rigidity) to resist the radially outward or expansive force exerted by the first arms 60 onto the fingers 150 without need for the lock tube 200 (or similar body).

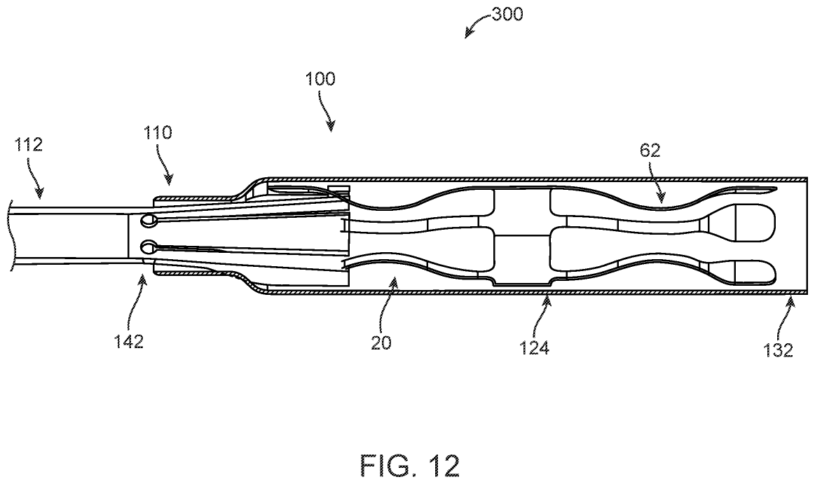

Regardless of whether the lock tube 200 is employed, one example of the interatrial shunting device 20 loaded to the delivery device 100 is reflected by FIG. 12. As a point of reference, the interatrial shunting device 20 and the delivery device 100 can be considered as combining to define a system 300 for treating a heart of a patient in accordance with principles of the present disclosure. In the view of FIG. 12, the delivery device 100 can be considered to be in a delivery condition, with the engagement unit 142 secured to the interatrial shunting device 20 in accordance with the descriptions above. Further, the engagement unit 142 is longitudinally arranged relative to the capsule 124 such that an entirety of the interatrial shunting device 20 is located within the capsule 124. With this construction, the capsule 124 exerts a radially compressive force onto the second arms 62. Where provided, the reinforcement layer 132 can further resist the outwardly expansive force being imparted by the second arms 62 onto the capsule 124. In the delivery condition of FIG. 12, the capsule 124 in combination with the engagement unit 142 secures and retains the interatrial shunting device 20 in the delivery state. In the delivery arrangement, the delivery device 100 can be manipulated by a user (e.g., the outer sheath assembly 110 and the inner shaft assembly 112 moved in tandem) to direct the interatrial shunting device 20 to a target location with the interatrial shunting device 20 held in the low profile delivery state. The interatrial shunting device 20 can then be released from the delivery device 20 by distally advancing the inner shaft assembly 112 (and thus the engagement unit 142) relative to the outer sheath assembly 110 (and thus the capsule 124) and/or vice-versa.

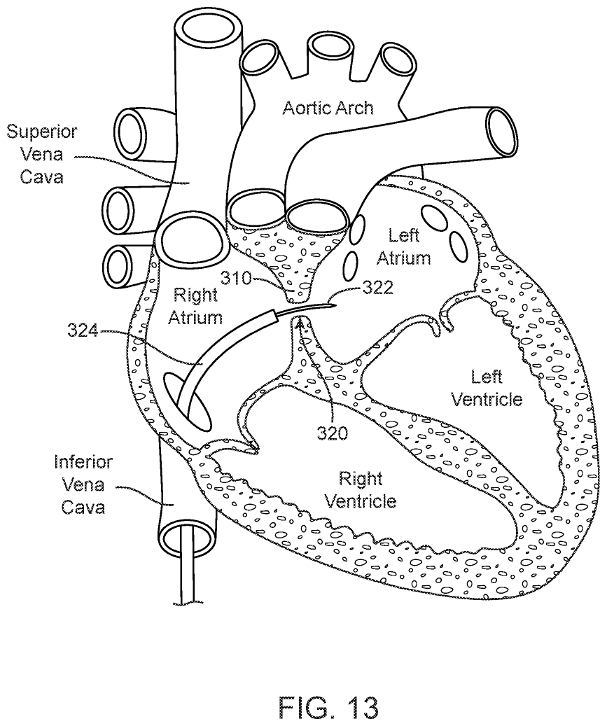

The interatrial shunt devices and heart treatment systems of the present disclosure can be utilized to address a variety of ailments, for example heart failure. In some examples, the methods of the present disclosure can include treating heart failure by establishing an interatrial shunt between the left and right atriums, for example to relieve pressure from the left atrium. For example, and with reference to FIG. 13, some methods of the present disclosure can include accessing the atrial septum 310 between the left and right atriums, and forming or puncturing a hole 320 through the atrial septum 310. The hole 320 can be established in a variety of fashions, and in some embodiments includes navigating a transseptal needle 322 (or similar device) through a delivery sheath or catheter 324 through the patient's vasculature and to the atrial septum 310 at the right atrium, such as by femoral, radial or brachial access. The transseptal needle 322 is then manipulated to pierce or puncture the hole 320 through the atrial septum 310 and then removed. The treatment system 300 (FIG. 12) is then advanced along a similar path to the atrial septum 310. For example, the delivery sheath 324 can remain in place, with the treatment system 300 being advanced there through. In other embodiments, a guide wire (not shown) can be inserted along the same pathway and the treatment system 300 then advanced over the guide wire.

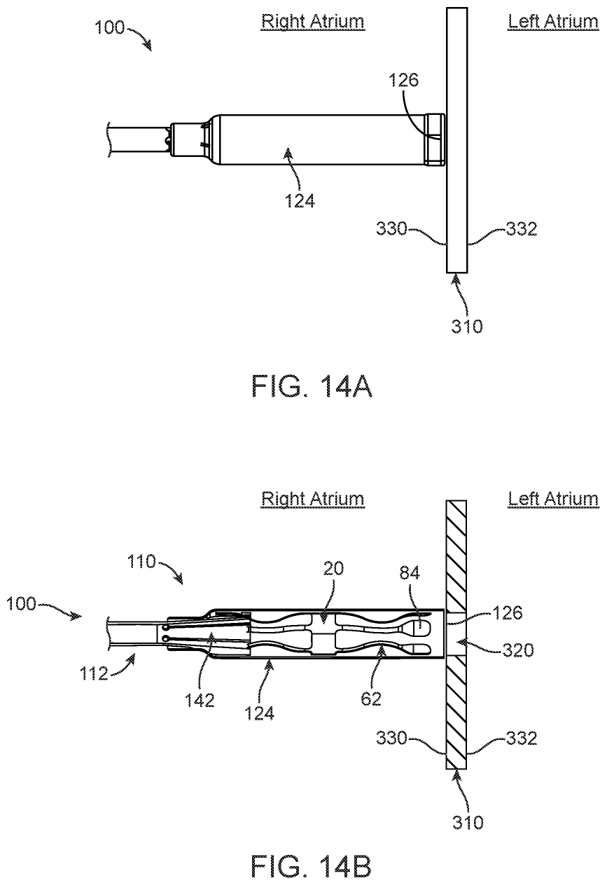

Regardless of the initial delivery technique, as shown in FIGS. 14A and 14B the delivery device 100 (loaded with the interatrial shunting device 20) is advanced, locating the capsule 124 in the right atrium and then arranging the distal end 126 in close proximity with the atrial septum 310, aligned with the hole 320. As a point of reference, the atrial septum 310 is shown in simplified form in FIGS. 14A and 14B for ease of understanding; further, only the capsule 124 of the outer sheath 120 (FIG. 4) is illustrated. The atrial septum 310 can be viewed as defining a first or right atrium side 330 opposite a second or left atrium side 332, with the hole 320 extending between and open to both of the sides 330, 332. In some embodiments, the optional the marker band 130 can assist the clinician in locating the distal end 126 relative to the hole 320.

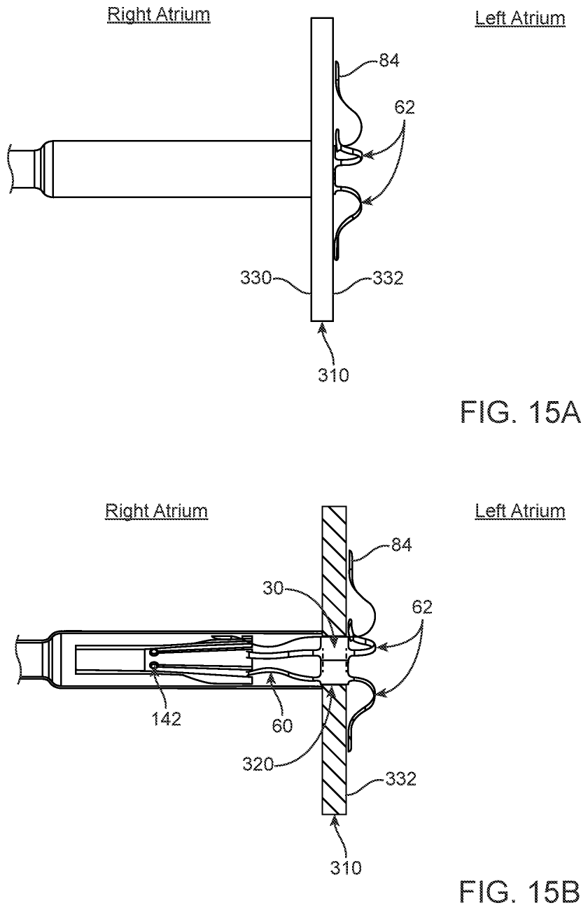

The inner shaft assembly 112 is distally advanced relative to the outer sheath assembly 110 (e.g., by operation of the handle assembly 114 (FIG. 4)), causing the engagement unit 142, and thus the interatrial shunting device 20 connected thereto, to slide distally relative to the distal end 126 of the capsule 124. With this distal advancement, the second arms 62 are progressively released or exposed relative to the distal end 126. The head 84 of each of the second arms 62 is caused to enter and pass through the hole 320. As the heads 84 move past the hole 320 (and into the left atrium), the second arms 62 begin to self-transition to the deployed state. As shown in FIGS. 15A and 15B, with further distal advancement of the engagement unit 142, the tube 30 becomes arranged within the hole 320 (identified generally in FIG. 15B). At this point in the method or procedure, the second arms 62 have self-transitioned or self-reverted to the deployment state, bringing the heads 84 into contact with the left atrium side 332; the first arms 60 remain connected to the engagement unit 142 and are retained in the delivery state.

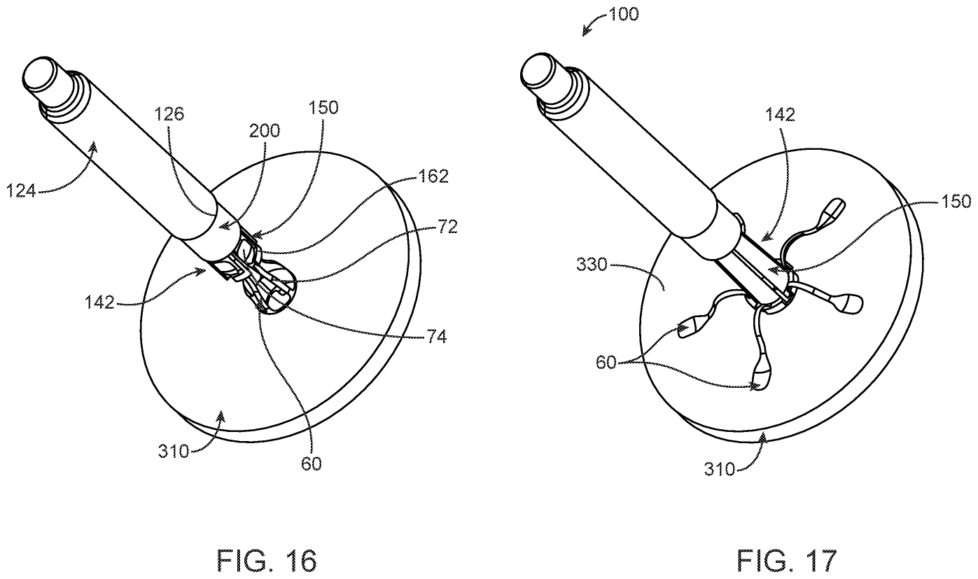

From the partial deployment arrangement of FIGS. 15A and 15B, the first arms 60 can be released in various manners, one example of which is reflected by FIGS. 16 and 17. In some embodiments, the outer sheath assembly 110 (FIG. 4) is retracted relative to the inner shaft assembly 112 (FIG. 4), sliding the capsule 124 along the engagement unit 142 (referenced generally) until the distal end 126 is proximal the heads 74 of the first arms 60 as shown in FIG. 16. FIG. 16 further reflects optional use of the lock tube 200. As shown, the lock tube 200 is located over a portion of the fingers 150, near, but proximal of, the first arms 60. With this configuration, the lock tube 200 assists in retaining the fingers 150, and thus the first arms 60, in the delivery state. With other embodiments in which the lock tube 200 (or similar component) is not included, the capsule 124 exhibits sufficient rigidity (and is located relative to the fingers 150) to perform this same function. The engagement unit 142 is then distally advanced toward the atrial septum 310 while the capsule 124 (and the lock tube 200, where provided) remain stationary. As the tab 162 slides distally along the intermediate segment 72 of the corresponding first arm 60, the first arms 60 each being to self-revert toward the deployed state in a controlled manner. FIG. 17 reflects a subsequent stage of the method or procedure. The engagement unit 142 has been distally advanced into close proximity with the right atrium side 330 of the atrial septum 310, allowing the first arms 60 to more fully self-transition or self-revert to the deployed state. The engagement unit 142 can then be rotated (e.g., counterclockwise relative to the orientation of FIG. 17) to release the first arms 60 from the fingers 150. Once released, the delivery device 100 can be withdrawn from the patient.

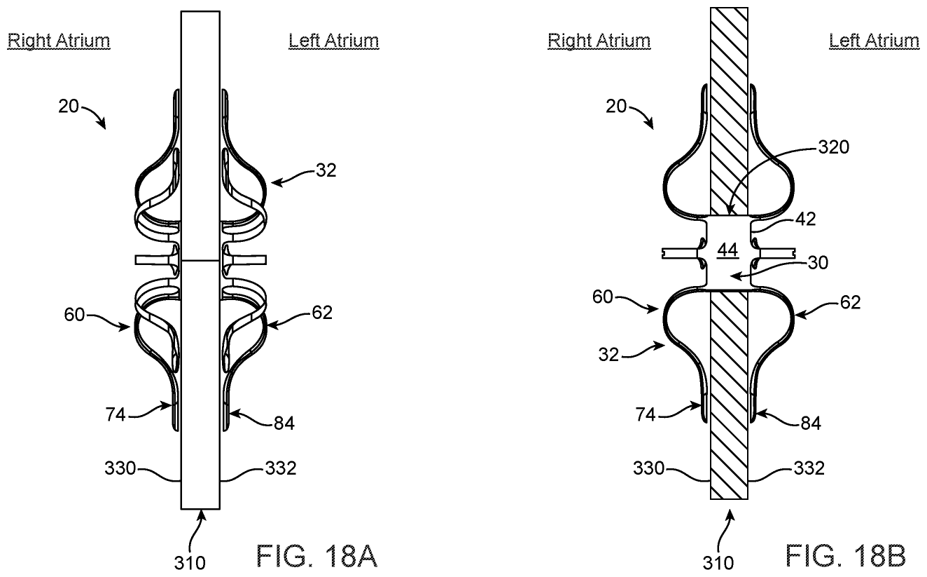

A number of other delivery tools and techniques can be employed to deliver and deploy the interatrial shunting devices of the present disclosure at an intended target location. Regardless, FIGS. 18A and 18B reflect one example of the interatrial shunting device 20 relative to the atrial septum 310 upon final implant. As shown, the tube 30 is located within the hole 320 (referenced generally), establishing a shunt across the atrial septum 310. The anchoring assembly 32 (referenced generally) secures the tube 30 relative to the atrial septum 310, and thus relative to the hole 320, for example by capturing a thickness of the atrial septum 310 (e.g., the heads 74, 84 of the first and second arms 60, 62 are naturally biased toward one another, pressing into the right atrium side 330 and the left atrium side 332, respectively).

Due to the length of the tube 30 closely approximating (e.g., being only slightly greater than) a thickness of the atrial septum 310 and the centered arrangement of the tube 30 as dictated by the anchoring assembly 32, projection of the tube 30 into the left atrium (and the right atrium) is minimal in some embodiments. With some examples, the second end 42 of the tube 30 projects less than 2 mm beyond the left atrium side 332 into the left atrium. With these and other arrangements, the implanted tube 30 beneficially presents minimal, if any, impediments to normal blood flow activity within the left atrium (e.g., exposure of the tube 30 in the left atrium is minimal in some embodiments to minimize risk of stroke). Regardless, following final implant, the lumen 44 is free of any foreign bodies, materials, mechanisms, etc. (e.g., the interatrial shunting device 20 does not include a valve, filter, etc., within the lumen 44). The solid wall construction of the tube 30 as described above impedes or prevents tissue ingrowth (or overgrowth) from the atrial septum 310 through a thickness of the tube 30 and into the lumen 44 over an extended period of time following implant. Thus, the lumen 44 will remain completely open for many months or years following implant, consistently providing the intended shunting effects.

The interatrial shunting devices, delivery devices, heart treatment systems and methods of the present disclosure provide a marked improvement over previous designs. The interatrial shunting devices of the present disclosure include a solid wall tube that prevents tissue overgrowth across the atrial septum, and can have minimal exposure of the device in the left atrium to minimize stroke and other risks. Anchoring assemblies provided with the interatrial shunting devices of the present disclosure can self-deploy from a compressed arrangement conducive to low profile, catheter-based delivery, and can be well-suited for use with simple and straightforward delivery devices.

Although the present disclosure has been described with reference to preferred embodiments, workers skilled in the art will recognize that changes can be made in form and detail without departing from the spirit and scope of the present disclosure.

What is claimed is:

1. An interatrial shunting device comprising:
a tube defining a first end opposite a second end, and a tube wall extending to and between the first and second ends, wherein the tube wall is a solid body and defines a lumen of the tube, the lumen being open at the first and second ends; and
an anchoring assembly carried by the tube and configured to secure the interatrial shunting device to a native atrial septum.

2. The interatrial shunting device of claim 1, wherein the interatrial shunting device is characterized by the absence of material within the lumen.

3. The interatrial shunting device of claim 1, wherein the tube wall is solid in transverse cross-section across an entire length of the tube from the first end to the second end.

4. The interatrial shunting device of claim 1, wherein the tube wall is configured to prevent tissue ingrowth from an exterior of the tube into the lumen.

5. The interatrial shunting device of claim 1, wherein the anchoring assembly is configured to be transitionable between a delivery state and a deployed state.

6. The interatrial shunting device of claim 5, wherein tube defines a longitudinal axis, and further wherein a footprint of the anchoring assembly in a plane transverse to the longitudinal axis in the delivery state is less than the footprint in the deployed state.

7. The interatrial shunting device of claim 6, wherein the anchoring assembly is configured to assume the delivery state in response to an applied external force, and is further configured to self-transition to the deployed state upon removal of the applied external force.

8. The interatrial shunting device of claim 7, wherein an outer diameter of the tube is substantially identical in the delivery state and the deployed state.

9. The interatrial shunting device of claim 5, wherein the anchoring assembly includes:
a first arm extending from the first end; and
a second arm extending from the second end;
wherein the anchoring assembly is configured to engage an atrial septum between the first and second arms in the deployed state.

10. The interatrial shunting device of claim 9, wherein the delivery state includes the first arm extending away from the first end in a first direction and the second arm extending away from the second end in a second direction, the first direction being opposite the second direction.

11. A system for treating a heart of a patient, the system comprising:
an interatrial shunting device including:
a tube defining a first end opposite a second end, and a tube wall extending to and between the first and second ends, wherein the tube wall is a solid body and defines a lumen of the tube, the lumen being open at the first and second ends,
an anchoring assembly carried by the tube and configured to secure the interatrial shunting device to a native atrial septum; and
a delivery device configured to retain the interatrial shunting device in a delivery state for delivery to a native atrial septum and to release the interatrial shunting device for implant at an opening in the native atrial septum in a deployed state.

12. The system of claim 11, wherein the delivery device includes:
an outer sheath assembly including a capsule; and
an inner shaft assembly slidably received within the outer sheath assembly and including an engagement unit;
wherein the delivery device is configured to provide a delivery condition in which the engagement unit is connected to the interatrial shunting device and the capsule encompasses and retains the interatrial shunting device in the delivery state.

13. The system of claim 12, wherein the anchoring assembly includes a first arm extending from the first end and a second arm extending from the second end, and further wherein the delivery condition includes the interatrial shunting device located within the capsule such that the second arm extends distally from the tube and the first arm extends proximally from the tube and is connected to the engagement unit.

14. The system of claim 13, wherein the delivery device is further configured to be transitionable from the delivery arrangement to a partial deployment arrangement in which the second arm is distal the capsule as self-reverts to the deployed state, and the first arm is within the capsule and is retained in the delivery state.

15. The system of claim 14, wherein the delivery device is further configured to be transitionable from the partial deployment arrangement to a released arrangement in which the first arm is distal the capsule and is released from the engagement unit to self-revert to the deployed state.

16. A method of treating heart failure, comprising:
forming a hole in an atrial septum of the heart; and
implanting an interatrial shunting device at the hole;
wherein the step of implanting includes:
positioning a tube of the interatrial shunting device within the hole, the tube defining a first end opposite a second end, and a tube wall extending to and between the first and second ends, wherein the tube wall is a solid body and defines a lumen of the tube, the lumen being open at the first and second ends,
arranging an anchoring assembly of the interatrial shunting device to engage the atrial septum and secure the tube within the hole.

17. The method of claim 16, wherein the method is characterized by the absence of tissue ingrowth across the lumen following the step of implanting.

18. The method of claim 16, wherein the method is characterized by the absence of a foreign body within the lumen following the step of implanting.

19. The method of claim 16, wherein the atrial septum separates left and right atriums of the heart and defines a left atrium side opposite a right atrium side, and further wherein following the step of implanting the first end is more proximate the right atrium side, the second end is more proximate left atrium side, and the second end projects less than 2 mm beyond the left atrium side into the left atrium.

20. The method of claim 16, wherein the anchoring assembly includes a first arm extending from the first end and a second arm extending from the second end, and further wherein the step of implanting includes:
advancing a delivery device to locate the tube within the hole, wherein the step of advancing includes the anchoring assembly retained in a delivery state with the first arm located proximal the second arm;
retracting an outer sheath assembly of the delivery device to expose the second arm, wherein the second arm self-transitions from the delivery state to a deployed state upon being released from the outer sheath assembly and is located adjacent a second side of the atrial septum;
further retracting the outer sheath to expose the first arm at location adjacent an opposite, first side of the atrial septum;
wherein the first arm self-transitions to a deployed state following the step of further retracting;
and further wherein in the deployed state, the first and second arms engage the first and second sides, respectively.
